(19) United States
(12) Patent Application Publication
(10) Pub. No.: US 2001/0055627 A1
(43) Pub. Date: Dec. 27, 2001

(54) Compositions And Methods For Regulating Lipoproteins And Hypercholesterolemia With Limonoids, Flavonoids And Tocotrienols

(76) Inventors: Najla Guthrie, London (CA); Elzbieta Maria Kurowska, London (CA)

Correspondence Address: DAVIDSON, DAVIDSON & KAPPEL, LLC, 485 SEVENTH AVENUE, 14TH FLOOR, NEW YORK, NY, 10018, US

(21) Appl. No.: 09/481,724

(22) Filed: Jan. 12, 2000

Related U.S. Application Data
(63) Continuation-in-part of application No. 08/938,640, filed on Sep. 26, 1997, now Pat. No. 6,251,400.

(51) Int. Cl. A61K 35/78

(52) U.S. Cl. 424/736; 424/725

(57) ABSTRACT

/Composition and methods for the prevention and treatment of hypercholesterolemia, hyperlipidemia and atherosclerosis are described. Individuals at a high risk of developing or having hypercholesterolemia and atherosclerosis undergoing conventional therapies may be treated with an effective dose of triperpene derivatives in limonoids, polyphenolic flavonoid compounds, tocotrienols or a combination of these agents.

### 1. CROSS-REFERENCE TO RELATED APPLICATIONS

This application is a Continuation-In-Part of co-pending application U.S. patent application Ser. No. 08/938,640, filed on Sep. 26, 1997, entitled “Compositions and Methods for Treatment of Neoplastic Diseases and Hypercholesterolemia with Citrus Limonoids and Flavonoids and Tocotrienols”, the entire disclosure of which is incorporated herein by reference.

### 2. BACKGROUND OF THE INVENTION

The present invention relates to compositions and methods for the prevention and treatment of atherosclerosis, hyperlipidemia and hypercholesterolemia, with limonoids, flavonoids and/or tocotrienols. Limonoids are a group of chemically related triterpene derivatives found in the Rutaceae and Meliaceae families. Citrus limonoids are among the bitter principals in citrus juices such as lemon, lime, orange and grapefruit. Flavonoids are polyphenolic compounds that occur ubiquitously in plant foods especially in orange, grapefruit and tangerine. Tocotrienols are present in palm oil and are a form of vitamin E having an unsaturated side chain. In the practice of the prevention and/or treatment of atherosclerosis, hyperlipidemia and/or hypercholesterolemia, the flavonoids, limonoids and tocotrienols are used to inhibit production of cholesterol, low-density lipoprotein (LDL) and apo-B protein. This invention is related to the discovery that citrus limonoids, flavonoids and/or tocotrienols in specific combinations, can reduce elevated serum cholesterol concentrations as well as apo-B In vivo. The invention further relates to the identification of compounds of the limonoids, flavonoids and trocotrienols that have the specific inhibitory effects on synthesis of liver cholesteryl esters and/or degradation of apo-B. Methods and compositions of these compounds in the treatment and prevention of atherosclerosis, hyperlipidemia and hypercholesterolemia are disclosed. The present invention has implications in the treatment of cardiovascular diseases.

### 2.1 Citrus Limonoids

Limonoids are a group of chemically related triterpene derivatives found in the Rutaceae and Meliaceae families. Limonoids are among the bitter principals found in citrus fruits such as lemons, lime, orange and grapefruit. They are also present as glucose derivatives in mature fruit tissues and seed, and are one of the major secondary metabolites present in citrus. Limonoids have been found to have anti-carcinogenic activity in laboratory animals. The furan moiety attached to the D-ring is specifically responsible for detoxifying of the chemical carcinogen by induction of the liver glutathione -S- transferase enzyme system (Lam, et al., 1994, Food Technol. 48:104-108).

Citrus fruit tissues and by-products of juice processing such as peels and molasses are sources of limonoid glucosides and citrus seeds contain high concentrations of both limonoid aglycones and glucosides. Limonoid aglycones in the fruit tissues gradually disappear during the late stages of fruit growth and maturation.

Thirty-eight limonoid aglycones have been isolated from citrus. The limonoids are present in thee different forms: the dilactone (I) is present as the open D-ring form (monolactone), the limonoate A-ring lactone (II) and the glucoside form (III). Only the monolactones and glucosides are present in fruit tissues. (Hasegawa S. Et al., 1994, in Food Phytochemicals for Cancer Prevention I, eds M-T. Huang et al., American Chemical Society, 198-207).

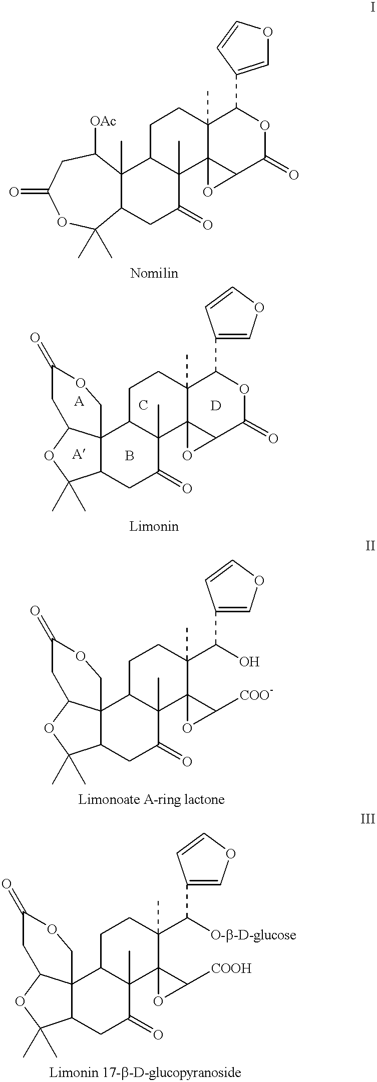

Compound III is the predominant limonoid glucoside found in all juice samples. In orange juice it comprises 56% of the total limonoid glucosides present, while in grapefruit and lemon juices, it comprises an average of 63% to 66% respectively. Procedures for the extraction and isolation of both aglycones and glucosides have been established to obtain concentrated sources of various limonoids (Lam, L. K. T. et al., 1994, in Food Phytochemicals for Cancer Prevention, eds. M. Huang, T. Osawa, C. Ho and R. T. Rosen, ACS Symposium Series 546, p 209). The use of limonoids alone or in combination with citrus flavonoid or tocotrienol has not been reported for the prevention and treatment of atherosclerosis, hyperlipidemia and hypercholesterolemia. Similarly, the use of limonoids from citrus juices alone or in combination with a citrus flavonoid, tocotrienol, a cholesterol lowering drug, or a combination of any one of these agents, has not been reported for the prevention and treatment of atherosclerosis, hyperlipidemia and hypercholesterolemia.

### 2.2 Citrus Flavonoids

Epidemiological studies have shown that flavonoids present in the Mediterranean diet reduce the risk of death from coronary heart disease (Hertog, M. G. Et al., 1993, Lancet: 342, 1007-1011). Soybean isoflavones for example, genistein, which is a minor component of soy protein preparations may have cholesterol-lowering effects (Kurowska, E. M. et al., 1990, J. Nutr. 120:831-836). The flavonoids present in citrus juices such as orange and grapefruit include, but are not limited to, hesperetin and naringenin respectively. The use of flavonoids from citrus juices alone or in combination with a citrus limonoid, tocotrienol, a cholesterol - lowering drug, or a combination of any one of these agents, has not been reported for the treatment of hypercholesterolemia.

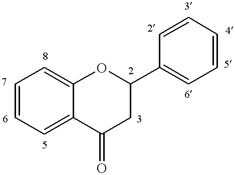

| | 5 | 7 | 3″ | 4″ |
|---|---|---|---|---|
| HESPERETIN | OH | OH | OH | OCH₃ |
| NARINGENIN | OH | OH | — | OH |

The flavonoids present in tangerine include, but are not limited to tangeretin or nobiletin.

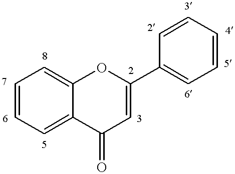

| | 5 | 6 | 7 | 8 | 4″ | 5″ |
|---|---|---|---|---|---|---|
| TANGERETIN | O CH₃ | O CH₃ | O CH₃ | O CH₃ | O CH₃ | — |
| NOBILETIN | O CH₃ | O CH₃ | O CH₃ | O CH₃ | O CH₃ | O CH₃ |

### 2.3 Tocotrienols in Palm Oil

Tocotrienols are present in palm oil and are a form of vitamin E having an unsaturated side chain. They include, but not limited to alpha-tocotrienol, gamma-tocotrienol or delta-tocotrienol.

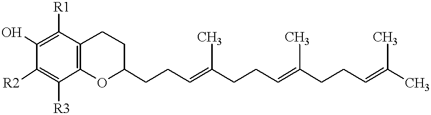

| | R1 | R2 | R3 |
|---|---|---|---|
| α-tocotrienol | CH₃ | CH₃ | CH₃ |
| γ-tocotrienol | H | CH₃ | CH₃ |
| δ-tocotrienol | H | H | CH₃ |

The present invention discloses novel compounds and their combinations that are effective in reducing hypercholesterolemia, hyperlipidemia and apo-B production, and thus, preventing the development of vascular disease which is manifested by increase in cholesterol metabolism.

### 3. SUMMARY OF THE INVENTION

The present invention is directed to compositions and a method for the prevention and/or treatment of cardiovascular diseases, which involves using a combination composition of limonoids, flavonoids and/or tocotrienols to treat an individual at high risk for, or suffering from hypercholesterolemia, hyperlipidemia and atherosclerosis.

The present invention is also directed to compositions and a method for the prevention and/or treatment of cardiovascular diseases which involves using a combination composition of limonoids and flavonoids to an individual at high risk for, or suffering from hypercholesterolemia, hyperlipidemia and atherosclerosis.

The present invention is also directed to compositions and a method for the prevention and/or treatment of cardiovascular diseases which involves using a combination composition of limonoids and tocotrienols to an individual at high risk for, or suffering from hypercholesterolemia, hyperlipidemia and atherosclerosis.

The present invention is also directed to compositions and a method for the prevention and/or treatment of cardiovascular diseases which involves using a combination composition of tocotrienols and flavonoids to an individual at high risk for, or suffering from hypercholesterolemia, hyperlipidemia and atherosclerosis.

The present invention is directed to a method for the prevention and/or treatment of cardiovascular disease, atheroslcerosis or hypercholesterolemia, that is, lower serum cholesterol, apo-B, LDL cholesterol, using a composition comprising flavonoids, limonoids or tocotrienols, to treat an individual at high risk of or suffering from cardiovascular disease, for example, atherosclerosis or hypercholesterolemia.

The present invention further provides methods to treat hypercholesterolemia, that is lower serum cholesterol, apo-B and LDL cholesterol, using a composition flavonoids, limonoids, tocotrienols, a cholesterol-lowering drug or a combination of these agents to treat an individual at high risk of or suffering from hypercholesterolemia.

### 4. BRIEF DESCRIPTION OF FIGURES

FIG. 1 depicts the design of the animal experiment.

FIG. 2 describes the effect of limonoids or flavonoids (given in fruit juices) on total and liprotein cholesterol concentrations in rabbits fed casein diet.

FIG. 3 describes the overall apo-B production in HepG2 cells exposed to increasing concentrations of naringenin (FIG. 3a) and hesperetin (FIG. 3b).

FIG. 4 describes ¹⁴C-acetate incorporation into cellular lipids treated with limonin, hesperetin or naringenin.

FIG. 5 describes the effect of limonin, hesperetin or naringenin on apo-B production In vitro.

### 5. DETAILED DESCRIPTION OF THE INVENTION

The method of the invention involves administering an effective dose of a citrus flavonoid alone or in combination with a citrus limonoid, tocotrienol or a cholesterol lowering drug, to an individual who is identified as being at enhanced risk for atherosclerosis, hyperlipidemia, cardiovascular disease or hypercholesterolemia and/or as having atherosclerosis, hyperlipidemia, cardiovascular disease or hypercholesterolemia, in order to prevent and treat hypercholesterolemia.

It may be that the ability of flavonoids, limonoids and/or tocotrienols to lower cholesterol, to inhibit liver cholesterol synthesis, inhibit LDL cholesterol and apo-B synthesis, contributes to their effectiveness in the reduction of atherosclerosis and hypercholesterolemia and lowering the risk of cardiovascular disease. These possible mechanisms of action are in no way meant to limit the scope of the invention and are presented purely for explanatory and/or illustrative purposes.

### 5.1 Atherosclerosis and Hypercholesterolemia

In the United States, the complications of arteriosclerosis account for about one half of all deaths and for about one third of deaths in persons between 35 and 65 years of age. Atherosclerosis, or the development of atheromatous plaques in large and medium-sized arteries, is the most common form of arteriosclerosis. Many factors are associated with the acceleration of atherosclerosis, regardless of the underlying primary pathogenic change, for example, age, elevated plasma cholesterol level, high arterial blood pressure, cigarette smoking, reduced high-density lipoprotein (HDL) cholesterol level, or family history of premature coronary artery disease.

Elevated levels of blood cholesterol are known to be one of the major risk factors associated with coronary heart disease, the leading cause of death in North America. Dietary intervention has been proven to play an important role in prevention and treatment of hypercholesterolemia. Current dietary recommendations focus on reduced intake of saturated fat and cholesterol but numerous studies also demonstrated a role of other common macro-and micronutrients, such as carbohydrates, protein and vitamins, in modulation of cholesterolemic responses (Charleux, J. L. Nutr. Rev. 1996 54: S109-S114). However, during recent years, a growing interest has also been shown in investigating possible cardioprotective effects of plant-derived food products and their minor nutritive and non-nutritive constituents (Cook, N. C.; Samman, S. J. Nutr. Biochem. 1996, 7:66-76).

The risk of death from coronary artery disease has a continuous and graded relations to total serum cholesterol levels greater than 180 mg/dl (Stamler, J. Et al., 1986, JAMA 2546:2823). Approximately one third of adults in the United States have levels that exceed 240 mg/dl and, therefore, have a risk of coronary artery disease that is twice that of people with cholesterol levels lower than 180 mg/dl.

Lipid transport system involves lipoproteins which transport cholesterol and triglycerides from sites of absorption and synthesis to sites of utilization. The lipoprotein surface coat contains the free cholesterol, phospholipids, and apolipoproteins, thus permitting these particles to be miscible in plasma as they transport their hydrophobic cargo. Apolipoprotein B (apo-B) is the principal protein of the cholesterol-carrying low density lipoproteins (LDL) and is the determinant for cellular recognition and uptake of LDL by the high affinity LDL receptor. Binding of apo-B to LDL receptors results in internalization and degradation of LDL, promoting the clearance of LDL from plasma and regulating intracellular cholesterol handling and biosynthesis.

Acceleration of atherosclerosis is principally correlated with elevation of LDL, or beta fraction, which is rich in cholesterol but poor in triglycerides. Elevation of HDL or alpha fraction, has a negative correlation with atherosclerosis (Castelli, W. P. et al., 1986, JAMA 256:2835). HDL exerts a protective effect and the ratio of total cholesterol to HDL cholesterol is a better predictor of coronary artery disease than the level of either alone. Total cholesterol levels are classified as being desirably (<200 mg/dl), borderline high (200-239 mg/dl), or high (>240 mg/dl) (Report of the National Education Program Expert Panel on Detection, Evaluation, and Treatment of High Blood Cholesterol in Adults, 1988, Arch. Intern. Med. 148:36).

Advances in the study of cholesterol metabolism and coronary disease have initiated an era of increased emphasis on preventive therapy. New guidelines for the detection and treatment of high blood cholesterol in adults recommend that patients with high cholesterol levels or with borderline-high levels and two or more additional risk factors should have a measurement of LDL. LDL cholesterol levels are then classified as borderline-high risk (130-159 mg/dl) or high risk (≧160 mg/dl). Dietary treatment is recommended for those patients with high-risk levels of LDL and for those with borderline-high risk levels who have two or more additional risk factors. Drug treatment is recommended for all patients with LDL levels greater than 189 mg/dl and for those patients with LDL cholesterol levels between 159 and 189 mg/dl who have two or more additional risk factors. Among the many drugs that have been used to reduce serum cholesterol levels are cholestyramine, colestipol, clofibrate, gemfibrozil and lovastatin. The use of flavonoids alone or in combination with citrus limonoids, tocotrienols or a cholesterol-lowering drug has not been reported for the treatment of hypercholesterolemia.

### 5.2 Dosage and Formulations

Citrus limonoids, citrus flavonoids or tocotrienols may be formulated into pharmaceutical preparations for administration to mammals for prevention and treatment of cardiovascular disease, hypercholesterolemia or atherosclerosis.

Many of the citrus limonoids, flavonoids or tocotrienols may be provided as compounds with pharmaceutically compatible counterions, a form in which they may be soluble.

The therapeutic compounds or pharmaceutical compositions may be administered intravenously, intraperitoneally, subcutaneously, intramuscularly, intrathecally, orally, rectally, topically or be aerosol.

Formulations suitable for oral administration include liquid solutions of the active compound dissolved in diluents such as saline, water or PEG 400; capsules or tablets, each containing a predetermined amount of the active agent as solid, granules or gelatin; suspensions in a approximate medium; and emulsions.

Formulations suitable for parenteral administration include aqueous and non-aqueous isotonic sterile solutions, which contain buffers, antioxidants and preservatives. The formulations may be in unit dose or multi-dose sealed containers.

Patient dosages for oral administration of citrus limonoids range from 1-500 mg/day, commonly 1-100 mg/day, and typically from 1-100 mg/day. Stated in terms of patient body weight, usual dosages range from 0.01-10 mg/kg/day, commonly from 0.01-2.0 mg/kg/day, typically from 0.01 to 2.0 mg/kg/day.

Patient dosages for oral administration of citrus flavonoids range from 200-5000 mg/day, commonly 1000-2000 mg/day, and typically from 500-1500 mg/day. Stated in terms of patient body weight, usual dosages range from 15-70 mg/kg/day, commonly from 15-30 mg/kg/day, typically from 7-21 mg/kg/day.

Patient dosages for oral administration of tocotrienols range from 1-1200 mg/day, commonly 1-100 mg/day, and typically from 1-60 mg/day. Stated in terms of patient body weight, usual dosages range from 0.01-20 mg/kg/day, commonly from 0.01-2.0 mg/kg/day, typically from 0.01 to 1/0 mg/kg/day.

A variety of delivery systems for the pharmacological compounds may be employed, including, but not limited to, liposomes and emulsions. The pharmaceutical compositions also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Furthermore, one may administer the agent in a targeted drug delivery system, for example, in a liposome coated with tumor-specific antibody. The liposomes will be targeted to and taken up selectively by the tumor.

In cases of local administration or selective uptake, the effective local concentration of the drug may not be related to plasma concentration.

The above disclosure generally describes the present invention. A more complete understanding can be obtained by reference to the following specific examples which are provided herein for purposes of illustration only, and are not intended to limit the scope of the invention.

### 6. EXAMPLE

### Hypocholesterolemic Effects of Naringenin and Hesperetin in Rabbits and HepG2 Cells

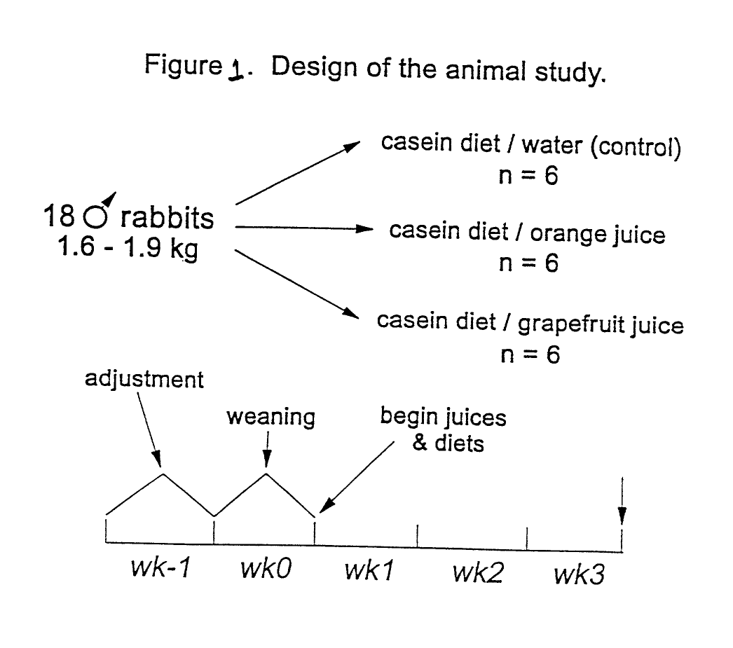

The effect of naringenin and hesperetin on casein-induced hypercholesterolemia was studied in rabbits. In each of the two experiments, rabbits were assigned to thee experimental groups. Each group was given semipurified, cholesterol-free casein diet and either water, orange juice or grapefruit juice to drink. The control group received water to drink and the experimental groups were given either orange juice or grapefruit juice (reconstituted concentrates made up to two times the regular strength). To ensure that the juice consumption did not affect the intake of other food components, diets given to the experimental groups were modified to compensate for additional sugar present in the juices and for any changes in the food intake observed during the study. FIG. 1 depicts the design of the experiment.

Dextrose in the diets of the hesperitin (orange juice) and naringenin (grapefruit juice) groups was reduced to account for the sugar consumed via citrus juices. All other dietary components were left unchanged. (Table 1).

TABLE 1
Average Percentage Composition of Cholesterol-free
Semipurified Casein Diets Fed to Rabbits

| | Diet | | |
|---|---|---|---|
| Ingredient | Control | Orange Juice | Grapefruit Juice |
| Casein | 27.0 | 35.4 | 30.3 |
| Dextrose | 55.7 | 46.4 | 50.2 |
| Alphacel | 4.8 | 5.0 | 5.4 |
| Salt mixture | 4.0 | 4.2 | 4.5 |
| (Phillips & Hart) | | | |
| Molasses | 4.2 | 4.5 | 4.7 |
| Palm oil | 1.3 | 1.4 | 1.5 |
| Soy Oil | 2.2 | 2.3 | 2.5 |
| Oil soluble vitamins | 0.5 | 0.6 | 0.6 |
| Water soluble vitamins | 1.5 | 1.6 | 1.7 |

Serum Analysis: At the end of the study, VLDL, LDL and HDL fractions were separated by discontinuous density gradient ultracentrifugation. Total cholesterol in serum and in each lipoprotein fraction were measured using the Boehinger Manheim CHOL kit (Manheim, Germany).

Liver Tissue Analysis: Total lipids were extracted from liver samples according to Foich et al., 1957, J. Biol. Chem. 226:497-509. Total and free cholesterol in lipid extracts were determined using the Boehinger Manheim kits.

Fecal Analysis: Feces collected for thee days during the last week of the study were freeze-dried. Fecal neutral steroids were extracted with petroleum ether, following saponification. Fecal cholesterol in the extracts was quantified using the CHOL kit. Fecal bile acids were extracted with t-butanol and enzymatically quantified with 3a-hydroxysteroid dehydrogenase (van der Meer et al., 1985, In Cholesterol Metabolism in Human Disease Studies in the Netherlands, eds. A. C. Beynen, et al., 113-119).

The effect of naringenin and hesperetin on metabolism of cholesterol in rabbits was studies using HepG2 cells In vitro.

Cell Culture: Confluent HepG2 cells were preincubated in minimum essential medium (MEM) containing 1.0% bovine serum albumin (BSA) for 24 hours. This was followed by a second 24 hour incubation with or without various non-toxic concentrations of naringenin or hesperitin (7.5-60 ug/ml). At the end of the second 24 hour incubation, the apo-B content of the media was determined by enzyme-lined immunosorbent assay (ELISA) (Young et al., 1986, Clin. Chem. 32:1484-1490).

¹⁴C-acetate Incorporation: To determine whether exposure to flavonoids can also alter intracellular lipid metabolism, confluent HepG2 cells were incubated for 24 hours with the highest apo-B-reducing, non-toxic concentration of these flavonoids, in the presence of ¹⁴C-acetate (0.5u Ci/ml medium). Radiolabelled lipids were then extracted from cells using heptane/isopropyl alcohol (3:2, v/v), and separated by thin layer chomatrography. A hexane/ethyl ether/acetic acid solvent (75:25:1, v/v) was used to separate cholesterol, cholesterol esters and triglycerides. Lipid fractions were scraped into scintillation vials and the radioactivity was measured using Beckman Scintillation Counter.

Protein Determination: The soluble cellular protein was extracted and measured using Pierce Coomassie Plus Protein Assay.

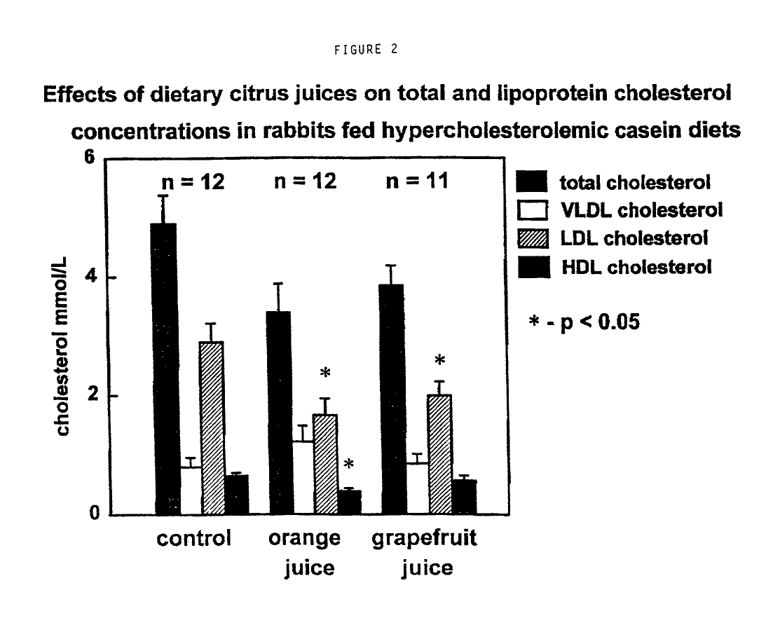

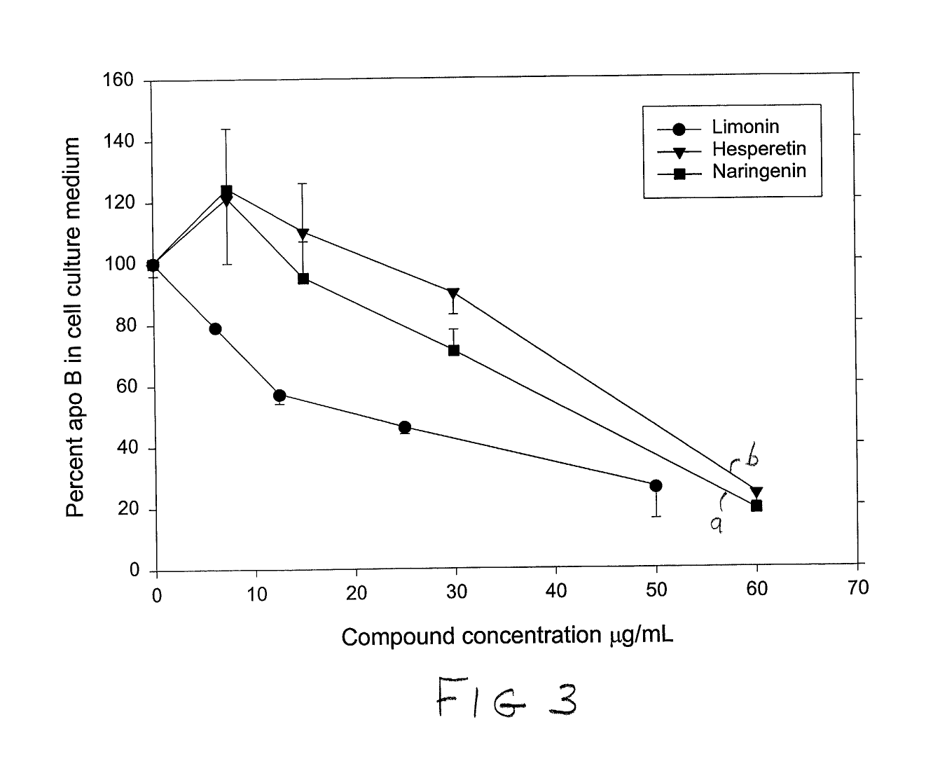

Results: (a) FIG. 2 describes the different lipoprotein concentrations of cholesterol after 3 weeks on naringenin or hesperitin supplementation in rabbits on a casein diet. Both reduced the LDL cholesterol levels in the animals whose diets were supplemented compared with LDL cholesterol levels in those given water (43% and 32% reduction for hesperitin and naringenin, respectively). This was associated with significant decreases in liver cholesterol esters (42% decrease in each supplemented group) but not with increases in fecal excretion of cholesterol and bile acids. The above data suggest that the reduction of LDL cholesterol was unlikely to be due to citrus pectins acting as cholesterol sequestrants in the intestine. Instead, the results indicated that changes in LDL cholesterol and in liver cholesterol esters might be induced by other juice components, such as limonoids and flavonoids, affecting metabolism of LDL in the liver.

The antihypercholesterolemia action of hesperetin, but not naringenin, is associated with reduction of liver cholesterol. (Table 2).

TABLE 2
Total Cholesterol, Cholesterol Esters and Free Cholesterol for Each
Experiment

| | Total Cholesterol | Cholesterol Esters | Free Cholesterol |
|---|---|---|---|
| Group (N) | (mg/g liver) | (mg/g liver) | (mg/g liver) |
| Control 12 | 3.8 ± 0.2^{a} | 1.2 ± 0.2^{c} | 2.7 ± 0.1 |
| Orange 12 | 3.1 ± 0.1^{a} | 0.7 ± 0.1^{d} | 2.4 ± 0.1 |
| Grapefruit 11 | 3.4 ± 0.2^{a} | 0.7 ± 0.1^{d} | 2.7 ± 0.1 |

Values are means ± SEM. a, b, c, d = significant different p < 0.05 (One-way ANOVA, Bonferroni test)

The effect of dietary hesperetin or naringenin on cholesterol levels is unlikely to be related to increased fecal excretion of cholesterol and bile acids since there was no difference in these measurements between the thee groups. (Table 3).

TABLE 3
Fecal cholesterol and bile acid excretion for each experimental group

| | | Cholesterol | Bile Acid |
|---|---|---|---|
| Group | (N) | (mg/day) | (mg/day) |
| Control | 12 | 3.0 ± 0.5^{a} | 10.5 ± 1.5 |
| Orange | 12 | 1.7 ± 0.3^{b} | 10.3 ± 1.9 |
| Grapefruit | 1 | 1.5 ± 0.3^{b} | 8.8 ± 1.1 |

Values are means ± SEM
^{a,b} = significant different p < 0.05 (One-way ANOVA)

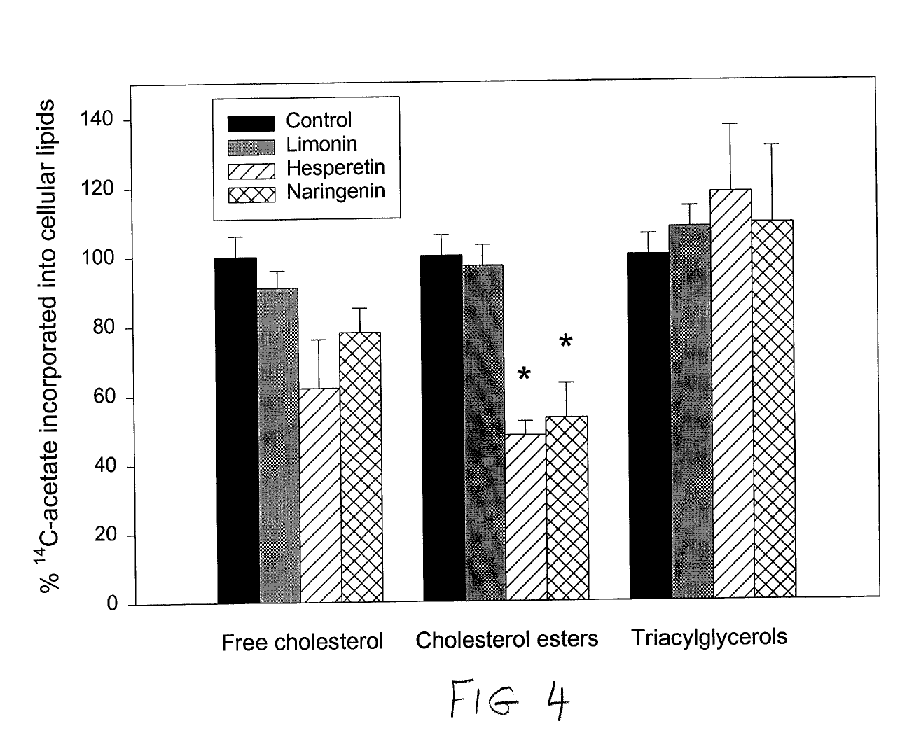

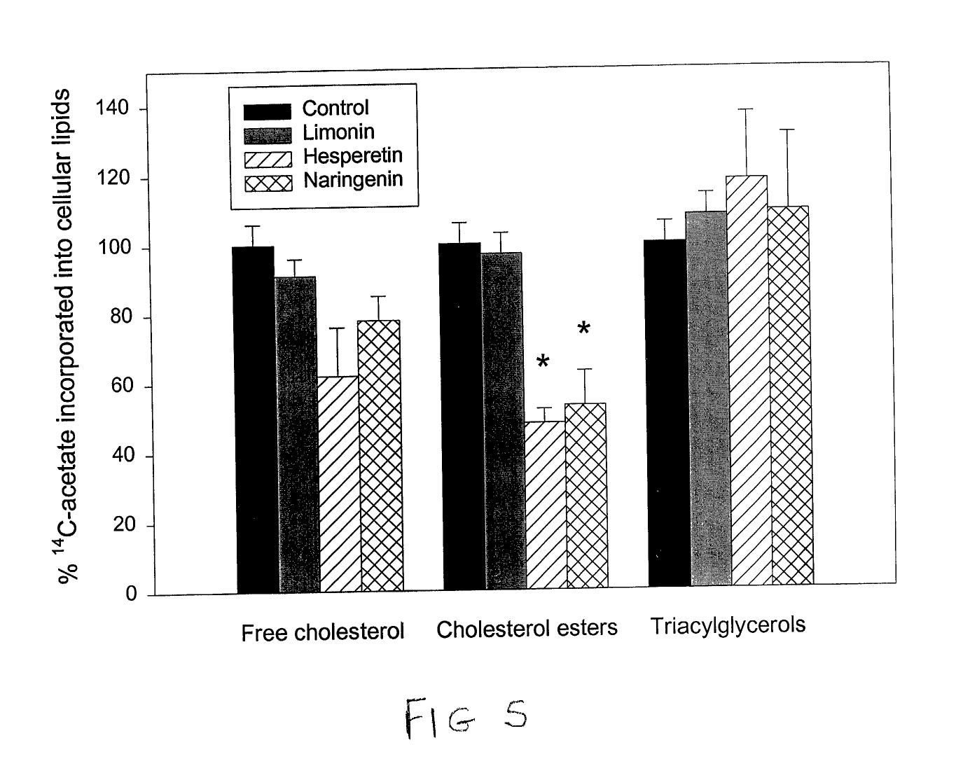

Naringenin and hesperitin present at high levels in citrus fruits have the ability to reduce overall production of apo-B in HepG2 cells. This effect is specific and not independent on reduction of cholesterol synthesis. FIGS. 3a, 3b, and 4. These results In vitro indicate that naringenin and hesperetin influence metabolism of lipoproteins directly in liver.

### 7. EXAMPLE

### Hypocholesterolemic Effects in HepG2 Cells

### 7.1: Limonoids

The limonoids, limonin and nomilin were examined for the cholesterol-lowering potential in HepG2 cells. Confluent HepG2 cells were preincubated for 24 h in a lipoprotein-free medium in which the fetal bovine serum was replaced by albumin to inhibit cell proliferation and to stimulate synthesis of cholesterol-containing lipoproteins. Cells were subsequently incubated for another 24 h in the same medium in the presence or absence of limonoids at the highest concentrations sustaining 100% cell viability, as determined by 3-(4,5-dimethylthiazol-2-yl)-2,5-diphenyltetrazolium bromide (MTT) viability assay. The limonoids were added to the cell culture medium after solubilization in dimethyl sulfoxide (DMSO). At the end of the incubation, culture media were collected and the concentration of the LDL structural protein, apo-B, was measured by Elisa by procedures already described in Section 6.0. Amount of lipoprotein-associated apo-B in the media (net apo-B secretion) was determined as described previously, calculated per mg cell protein and expressed as percent of control.

The results show that limonin and nomilin induced either a substantial or moderate reduction of apo-B levels. Limonin was the most active, reducing apo-B by 74% when added to cells at a concentration of 50 μg/mL. At the same concentration, nomilin reduced medium apo-B levels by 40%.

### 7.2 Flavonoids

Apo-B productioon (the structural protein of LDL) was studied in HepG2 cells, using a 24. The effect of flavonoids on apo-B production after 24 h incubation of cells and measuring apo-B by Elisa as described above. All flavonoids, reduced the apo-B levels dose dependently. IC₅₀ concentrations were 2.5 μg/ml for tangeretin, 4.9 μg/ml for nobiletin, 43.0 μg/ml for hesperetin and 48.5 μg/ml for naringenin.

### 7.3 Comparison of Limonoids and Flavonoids

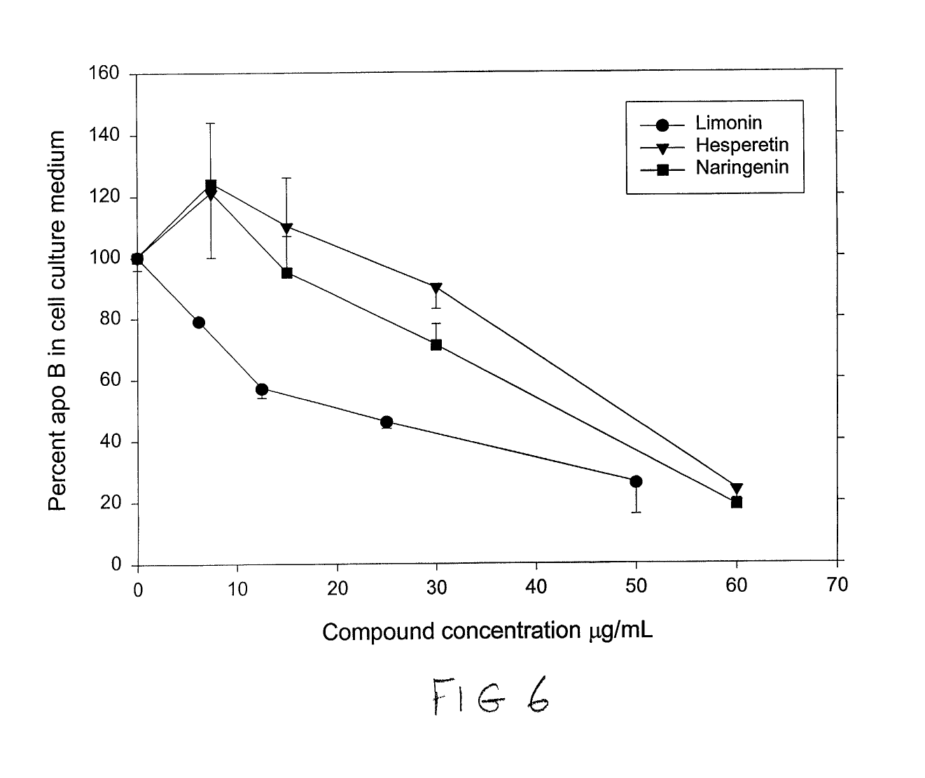

The mechanism by which limonin or hesperetin and naringenin exert their apo-B lowering effect in cells was compared in HepG2 cells. Limonin, like hesperetin and naringenin, reduced the apo-B content in a dose-dependent manner. However, the effect of limonin was greater than that of the flavonoids. The IC₅₀ concentration was 2-2.4 times lower for limonin than for both flavonoids (20.5 μm/L vs. 43.0 μm/L and 48.5 μm/L for limonin, hesperetin and naringenin, respectively). See FIG. 5.

To determine whether exposure to limonin altered intracellular lipid metabolism, confluent HepG2 cells were incubated for 24 h with or without the highest non-cytotoxic concentration of limonin. During the last 5 h of the incubation, ¹⁴C-acetate (0.5 μCi/mL) was added to the medium. The extraction and separation of cellular lipids was done described above.

FIG. 4 describes the results. At the highest non-toxic concentration (50 μg/ml), limonin had no effect on ¹⁴C-acetate incorporation into cellular cholesterol, cholesteryl esters and triacylglycerols. However, both flavonoids added to cells at the highest non-toxic levels (60 μg/ml) induced a significant, approximately 50% decrease in the incorporation of ¹⁴C-acetate into cellular cholesteryl esters without causing significant changes in label incorporation into free cholesterol and triacylglycerols. The results obtained suggest that the mechanism by which limonin lowers medium apo-B is different from that for flavonoids which were found to reduce the availability of neutral lipids that are required for the assembly of apo-B-containing lipoproteins. In contrast, the apo-B lowering action of limonin may be mediated via increases in the intracellular degradation of apo-B prior to lipoprotein assembly and secretion or via up regulation of LDL receptors responsible for catabolism of apo-B-containing lipoproteins.

### 8. EXAMPLE

### Hypocholesterolemic Effects of Flavonoids and Tocotrienols, alone or in Combinations, in a Hamster Model of Hypercholesterolemia

Extracted flavonoids and a tocotrienol-rich preparation from palm oil, all provided by KGK Synergize Inc. London, Ontario, were tested in the hamster model of experimental hypercholesterolemia. The purpose of the study was to establish whether in this model, 2% supplementation of casein diet with palm tocotrienol preparation containing 70% of pure tocotrienols α-, γ- and δ-(TTP), or a similar 2% supplementation with flavonoids, hesperidin extrated from oranges and naringin extrated from grapefruit, could lower the elevated LDL cholesterol levels induced by casein. A further purpose of the study was to determine whether supplementation with 2% TTP in combination with 2% hesperidin or 2% naringin could result in synergistic cholesterol-lowering effects. Synergistic cholesterol-lowering responses have been observed for combinations of tocotrienols and citrus flavonoids in human liver cell line HepG2. The additional goal of this study was to establish whether in the same hamster model of hypercholesterolemia, 0.13% supplementation of casein diet with a mixture of polymethoxylated flavones from tangerines (PMF) could lower the elevated LDL cholesterol levels induced by casein. This experiment was based on previous tests In vitro using human liver cell line HepG2, in which principal flavonoids from PMF, tangeretin and nobiletin, had much greater cholesterol-lowering potential than hesperetin or naringenin.

In hamsters, feeding a semipurified, casein-based, cholesterol-enriched diet for the period of 5 weeks induces experimental hypercholesterolemia associated with elevation of LDL cholesterol, similar to that observed in humans.

### Animals and Diets

Fifty four male Golden Syrian hamsters weighing 100-120 g at arrival were housed individually in a constant temperature 21-24° C. and with 12 h light:dark cycle. The animals were fed ground chow diet for 1 week. Thereafter, they were divided into seven groups (six groups of 8 animals and one group of 6 animals), each with similar average body weight, and fed experimental diets for 35 days. Food and water were provided at libitum. Food consumption was estimated during the last two weeks of treatment, for thee consecutive days during each week.

The percent composition of semipurified diets is presented in Table 4. The basal composition was similar to that described earlier. All diets contained 25% casein and 0.1% cholesterol. Experimental diets contained TTP and/or principal flavonoids, hesperidin or naringenin, added at indicated levels at the expense of rice flour.

TABLE 4
Percent Composition of Experimental Diets

| | | | | | 2% TTP + | 2% TTP + | |
|---|---|---|---|---|---|---|---|
| | | 2% | 2% | 2% | 2% | 2% | 0.13% |
| Ingredients | Control | TTP | hesperidin | naringin | hesperidin | naringen | PMF |
| Casein | 26.3 | 26.3 | 26.3 | 26.3 | 26.3 | 26.3 | 26.3 |
| Rice flour | 39.8 | 37.8 | 37.8 | 37.8 | 35.8 | 35.8 | 39.7 |
| Coconut oil | 10.0 | 10.0 | 10.0 | 10.0 | 10.0 | 10.0 | 10.0 |
| Safflower oil | 2.0 | 2.0 | 2.0 | 2.0 | 2.0 | 2.0 | 2.0 |
| Cellulose | 7.5 | 7.5 | 7.5 | 7.5 | 7.5 | 7.5 | 7.5 |
| Wheat bran | 7.5 | 7.5 | 7.5 | 7.5 | 7.5 | 7.5 | 7.5 |
| Choline chloride | 0.3 | 0.3 | 0.3 | 0.3 | 0.3 | 0.3 | 0.3 |
| Potassium bicarbonate | 2.0 | 2.0 | 2.0 | 2.0 | 2.0 | 2.0 | 2.0 |
| Vitamin mix | 1.0 | 1.0 | 1.0 | 1.0 | 1.0 | 1.0 | 1.0 |
| Mineral mix | 3.5 | 3.5 | 3.5 | 3.5 | 3.5 | 3.5 | 3.5 |
| Cholesterol | 0.1 | 0.1 | 0.1 | 0.1 | 0.1 | 0.1 | 0.1 |
| Supplement | — | 2.0 | 2.0 | 2.0 | 4.0 | 4.0 | 0.13 |

### Serum Analysis

At the end of the study, the animals were fasted overnight and blood samples were taken by heart puncture. Total cholesterol in whole serum and in HDL fraction as well as total triacylglycerols were measured by enzymatic timed-endpoint methods, using the Beckman Coulter reagents (CHOL Reagent, HDLD HDL Reagent and TG Triglycerides GPO Reagent, respectively). All measurements were performed using SYNCHON LX System. VLDL+LDL cholesterol concentrations were calculated as a difference between total and HDL cholesterol. Results obtained were analyzed by one-way ANOVA followed by Dunnet's or Student-Newman-Keuls test for all groups and additionally by t-test for the PMF vs. the control group. Significant differences were reported at p<0.05.

### Results

Table 5 shows that growth rates were similar between the groups. Food consumption was significantly higher in the control only in animals fed 2% naringenin.

TABLE 5
Growth performance of hamsters fed experimental

| | | | Food |
|---|---|---|---|
| | Initial Weight | Growth Rate | Consumption |
| Diet | (g) | (g/day) | (g/day) |
| Control | 105.50 ± 6.95 | 1.10 ± 0.35 | 8.69 ± 1.43 |
| 2% TTP | 106.09 ± 9.16 | 1.00 ± 0.33 | 8.59 ± 1.21 |
| 2% Hesperidin | 107.01 ± 4.78 | 0.98 ± 0.24 | 13.15 ± 4.72* |
| 2% Naringin | 106.50 ± 5.49 | 1.03 ± 0.26 | 10.44 ± 2.36 |
| 2% TTP + 2% hesperidin | 106.88 ± 4.24 | 0.67 ± 0.36 | 10.25 ± 1.79 |
| 2% TTP + 2% naringin | 105.69 ± 9.83 | 0.91 ± 0.20 | 9.09 ± 1.35 |
| 0.13% PMF | 106.75 ± 5.19 | 0.84 ± 0.33 | 8.11 ± 0.53 |

Values are means ± SD. Six hamsters in 0.13% PMF group. Eight hamsters in each of the remaining groups. *significantly different from control group by ANOVA followed by Dunnett's test.

Effects of dietary treatments on serum lipid responses are presented in Table 6. After five weeks of feeding experimental diets, hamsters fed the control diet developed hypercholesterolemia associated with elevation of VLDL+LDL cholesterol. Supplementation with 2% TTP significantly reduced serum total, VLDL+LDL and HDL cholesterol as well as VLDL/HDL cholesterol ratio (by 37%, 52%, 26% and 65%, respectively). Less substantial hypolipidemic effects were observed in animals fed 2% hesperidin or naringin. Addition of 2% hesperidin did not induce significant changes in any of the blood lipids but tended to lower serum total triacylglycerols (36% reduction). Addition of 2% naringin significantly reduced serum total and HDL cholesterol (each by 11%) without significant altering VLDL+LDL cholesterol and total triacylglycerols. Supplementation with TTP plus hesperidin resulted in a significant reduction of serum total, VLDL+LDL and HDL cholesterol. The effect of TTP combined with hesperidin was similar to that observed for TTP alone, except, the reduction of HDL cholesterol induced by the combination was significantly greater than that produced by either TTP or hesperidin (32% vs, 26% and 7%, respectively). A combination of TTP and naringin also caused significantly greater decreases in HDL cholesterol than TTP or naringin alone (39% vs. 26% and 11%, respectively), but also tended to cause greater reduction of serum total cholesterol (43% vs. 37% and 11%, respectively) (p<0.05 by Student-Newman-Keuls test).

A 0.13% supplementation of casein diet with PMF significantly reduced concentrations of serum total, VLDL+LDL and HDL cholesterol (by 13%, 17% and 10%, respectively). It also significantly lowered serum total triacylglycerols by 43%.

TABLE 6
Changes in serum lipid profiles in hamsters fed experimental diets

| | | | | | | |
|---|---|---|---|---|---|---|
| | | Total | VLDL + | HDL | VLDL + | |
| I. | | choles- | LDL Cho- | cho- | LDL/HDL | Total tria- |
| II. | | terol | lesterol | lesterol | cholesterol | cylglycerols |
| III. | DIET | mM/L | mM/L | mM/L | ratio | mM/L |

| | | | | | |
|---|---|---|---|---|---|
| Control | 7.40 ± | 3.02 ± | 4.38 ± | 0.69 ± | 4.15 ± 1.68 |
| | 0.73 | 0.53 | 0.35 | 0.12 | |
| 2% TPT | 4.67 ± | 1.44 ± | 3.23 ± | 0.45 ± | 3.46 ± 0.84 |
| | 0.37* | 0.37* | 0.25* | 0.14* | |
| Percent change | −37% | −52% | −26% | | |
| 2% hesperidin | 6.73 ± | 2.66 ± | 4.07 ± | 0.66 ± | 2.64 ± 1.47 |
| | 0.66 | 0.40 | 0.38 | 0.09 | |
| Percent change | −9% | −12% | −7% | | −36% |
| 2% naringin | 6.56 ± | 2.66 ± | 3.89 ± | 0.68 ± | 3.58 ± 1.40 |
| | 1.09 | 0.59 | 0.59 | 0.12 | |
| Percent change | −11% | −12% | −11% | | |
| 2% TTP + | 4.72 ± | 1.76 ± | 2.96 ± | 0.60 ± | 3.42 ± 1.13 |
| 2% hesperidin | 0.51* | 0.17* | 0.37* | 0.05 | |
| Percent change | −36% | −42% | −32% | | |
| 2% TTP + | 4.20 ± | 1.54 ± | 2.65 ± | 0.60 ± | 3.34 ± 1.27 |
| 2% naringin | 0.69* | 0.31* | 0.53* | 0.17 | |
| Percent change | −43% | −49% | −39% | | |
| 0.13% PMF | 6.46 ± | 2.50 ± | 3.96 ± | 0.63 ± | 2.37 ± 0.46 |
| | 0.54^{#} | 0.26^{#} | 0.34^{#} | 0.06 | |
| Percent change | −13% | −17% | −10% | | −43% |

Values are means ± SD. Six animals in PMF group, eight animals in each other group. *significantly different from control, within the row, by ANOVA followed by Dunnett's test, p < 0.05. ^{#}significantly different from the control value within the row by t-test.

The overall results showed that:

a) 2% dietary supplementation with TTP preparation containing 70% of pure tocotrienols had a profound effect on serum total and lipoprotein cholesterol, especially on VLDL+LDL cholesterol, which was reduced by more than 50%. The potency of TTP preparation used in the present invention was much greater than that observed earlier for any tocotrienol fraction, and comparable to that reported for hamsters given cholesterol-lowering drugs. This unexpectedly potent effect of TTP in the present invention may be due to its relatively high level in the diet as well as its unique composition. Previous tocotrienol-rich preparations, although usually used at higher levels, often contained up to 30% of alpha-tocopherol, which has been shown to attenuate the cholesterol suppressive action of tocotrienols in animals and in cells.

b) Supplementation with 2% naringin produced much less pronounced cholesterol-lowering responses than TTP, reducing both total and HDL cholesterol by 11%. Similar trends were observed for 2% hesperidin but its effects were relatively weak and not statistically significant. In the same animal model, a mixture of citrus flavonoids containing largely naringin and hesperidin (0.05% of each per 100 g diet) caused a 36% reduction of plasma total cholesterol. This was not associated with decreases in HDL cholesterol but HDL cholesterol concentrations reported in this particular study were unusually low

c) Combinations of TTP plus hesperidin or TTP plus naringin added to the hamsters' diet both caused more pronounced cholesterol-lowering effects than TTP, hesperidin or naringin alone, suggesting synergistic effects. These synergistic results are consistent with those obtained in HepG2 cells suggesting that tocotrienols and flavonoids modulate cholesterol metabolism via different mechanisms. Tocotrienols have been reported to act by inhibiting cholesterol biosynthesis pathway, and also, by increasing intracellular degradation of apo-B prior to assembly and secretion of apo-B-containing lipoproteins. In contrast, citrus flavonoids were demonstrated to act mainly by suppressing cellular synthesis of cholesteryl esters and/or inhibit HMGCoA reductase, the rate-limiting enzyme of cellular cholesterol synthesis. The synergy between TTP and hesperidin, and between TTP and naringin has been observed in spite of the fact that at 2% supplementation, dietary TTP preparation was much more potent than hesperidin or naringin.

d) In hamsters, 0.13% supplementation with PMF produced significant reductions in serum total, VLDL+LDL and HDL cholesterol, as well as in serum total triacylglycerols (13%, 17%, 10% and 43%, respectively). The hypolipidemic effects of PMF were equal or greater than those produced by hesperidin or naringin added to diets at much higher levels. The substantial cholesterol-lowering potential of PMF was consistent with results showing that in HepG2 cells, two principal PMF constituents, tangeretin and nobiletin, showed much greater apo-B-lowering activities than hesperetin or naringenin (IC₅₀'s 2.5 μg/ml and 4.9 μg/ml, respectively). In HepG2 cells, synergistic apo-B-lowering effects were also observed when either tangeretin or nobiletin were tested in combinations with tocotrienol rich fractions. The potent hypolipidemic responses observed in PMF-fed hamsters suggest that PMF should be considered as a component of cholesterol-lowering formulations in nutraceuticals supplements and in functional foods

The present invention is not to be limited in scope by embodiments disclosed in the examples which are intended as an illustration of one aspect of the invention and any methods which are functionally equivalent are within the scope of the invention. Indeed, various modifications of the invention in addition to those shown and described herein will become apparent to those skilled in the art from the foregoing description. Such modifications are intended to fall within the scope of the appended claims.

Various publications are cited herein, the disclosures of which are incorporated by reference in their entireties.

What is claimed is:

1. A pharmaceutical composition suitable for administering to a human subject at risk for or suffering from hypercholesterolemia, said composition comprising a cholesterol lowering effective amount of a limonoid selected from the group consisting of limonin and nomilin, and a flavonoid selected from the group consisting of hesperidin, naringin, naringenin, hesperitin, nobiletin and tangeretin.

2. A pharmaceutical composition suitable for administering to a human subject at risk for or suffering from hypercholesterolemia, said composition comprising a cholesterol-lowering effective amount of a limonoid selected from the group consisting of limonin and nomilin, and a tocotrienol.

3. A pharmaceutical composition suitable for administering to a human subject at risk for or suffering from hypercholesterolemia according to claim 2, said composition further comprising a cholesterol-lowering effective amount of a flavonoid selected from the group consisting of hesperidin, naringin, naringenin, hesperitin, nobiletin and tangeretin.

4. A pharmaceutical composition suitable for administering to a human subject at risk for or suffering from hypercholesterolemia, said composition comprising a cholesterol lowering effective amount of a flavonoid selected from the group consisting of hesperidin, naringin, naringenin, hesperitin, nobiletin and tangeretin and a tocotrienol.

5. The pharmaceutical composition according to claims 2, 3 or 4, wherein the tocotrienol is selected from the group consisting of alpha-tocotrienol, gamma tocotrienol, and delta-tocotrienol.

6. A method of treating hypercholesterolemia comprising administering to an individual in need thereof a pharmaceutical composition comprising cholesterol lowering effective amounts of a limonoid selected from the group consisting of limonin and nomilin, and a flavonoid selected from the group consisting of hesperidin, naringin, naringenin, hesperitin, nobiletin and tangeretin

7. The method according to claim 6, wherein the amount of the limonoid administered is in the range of 1 to 500 mg/day and the amount of the flavonoid is in the range of 200 to 5000 mg/day.

8. The method according to claim 6, further comprising administering a cholesterol lowering effective amount of a tocotrienol selected from the group consisting of alpha-tocotrienol, gamma tocotrienol, and delta-tocotrienol.

9. The method according to claim 8, wherein the amount of the tocotrienol is in the range of 1 to 1200 mg/day.

10. A method of treating hypercholesterolemia comprising administering to an individual in need thereof a pharmaceutical composition comprising a cholesterol lowering effective amount of a limonoid selected from the group consisting of limonin and nomilin, and a tocotrienol selected from the group consisting of alpha-tocotrienol, gamma tocotrienol, and delta-tocotrienol.

11. A method of treating hypercholesterolemia comprising administering to an individual in need thereof a pharmaceutical composition comprising a cholesterol lowering effective amount of a tocotrienol selected from the group consisting of alpha-tocotrienol, gamma tocotrienol, and delta-tocotrienol and a flavonoid selected from the group consisting of hesperidin, naringin, naringenin, hesperitin, nobiletin and tangeretin.

12. A method of treating hypercholesterolemia comprising administering to an individual in need thereof a pharmaceutical composition comprising a cholesterol lowering effective amount of nobiletin.

13. A method of treating hypercholesterolemia comprising administering to an individual in need thereof a pharmaceutical composition comprising a cholesterol lowering effective amount of tangeretin.

14. A pharmaceutical composition suitable for administering to a human subject at risk for or suffering from atherosclerosis, said composition comprising an anti-atherosclerotic lowering effective amount of a limonoid selected from the group consisting of limonin and nomilin, and a flavonoid selected from the group consisting of hesperidin, naringin, naringenin, hesperitin, nobiletin and tangeretin.

15. A pharmaceutical composition suitable for administering to a human subject at risk for or suffering from atherosclerosis, said composition comprising an anti-atherosclerotic effective amount of a limonoid selected from the group consisting of limonin and nomilin, and a tocotrienol.

16. A pharmaceutical composition suitable for administering to a human subject at risk for or suffering from atherosclerosis according to claim 2, said composition further comprising an anti-atherosclerotic effective amount of a flavonoid selected from the group consisting of hesperidin, naringin, naringenin, hesperitin, nobiletin and tangeretin.

17. A pharmaceutical composition suitable for administering to a human subject at risk for or suffering from atherosclerosis, said composition comprising an anti-atherosclerotic effective amount of a flavonoid selected from the group consisting of hesperidin, naringin, naringenin, hesperitin, nobiletin and tangeretin and a tocotrienol.

18. The pharmaceutical composition according to claims 2, 3 or 4, wherein the tocotrienol is selected from the group consisting of alpha-tocotrienol, gamma tocotrienol, and delta-tocotrienol.

19. A method of treating atherosclerosis comprising administering to an individual in need thereof a pharmaceutical composition comprising an anti-atherosclerotic effective amounts of a limonoid selected from the group consisting of limonin and nomilin, and a flavonoid selected from the group consisting of hesperidin, naringin, naringenin, hesperitin, nobiletin and tangeretin

20. The method according to claim 6, wherein the amount of the limonoid administered is in the range of 1 to 500 mg/day and the amount of the flavonoid is in the range of 200 to 5000 mg/day.

21. The method according to claim 6, further comprising administering an anti-atherosclerotic effective amount of a tocotrienol selected from the group consisting of alpha-tocotrienol, gamma tocotrienol, and delta-tocotrienol.

22. The method according to claim 8, wherein the amount of the tocotrienol is in the range of 1 to 1200 mg/day.

23. A method of treating atherosclerosis comprising administering to an individual in need thereof a pharmaceutical composition comprising an anti-atherosclerotic effective amount of a limonoid selected from the group consisting of limonin and nomilin, and a tocotrienol selected from the group consisting of alpha-tocotrienol, gamma tocotrienol, and delta-tocotrienol.

24. A method of treating atherosclerosis comprising administering to an individual in need thereof a pharmaceutical composition comprising an anti -atherosclerotic effective amount of a tocotrienol selected from the group consisting of alpha-tocotrienol, gamma tocotrienol, and delta-tocotrienol and a flavonoid selected from the group consisting of hesperidin, naringin, naringenin, hesperitin, nobiletin and tangeretin.

25. A method of treating atherosclerosis comprising administering to an individual in need thereof a pharmaceutical composition comprising an anti-atherosclerotic effective amount of nobiletin.

26. A method of treating atherosclerosis comprising administering to an individual in need thereof a pharmaceutical composition comprising an anti-atherosclerotic effective amount of tangeretin.

27. A method of treating hypercholesterolemia comprising administering to an individual in need thereof a pharmaceutical composition comprising a cholesterol lowering effective amount of limonoid selected from the group consisting of limonin and nomilin.

28. A method of treating atherosclerosis comprising administering to an individual in need thereof a pharmaceutical composition comprising an anti-atherosclerotic effective amount of limonoid selected from the group consisting of limonin or nomilin.
